(19) United States
(12) Patent Application Publication
(10) Pub. No.: US 2025/0197902 A1
(43) Pub. Date: Jun. 19, 2025

(54) CHEMOENZYMATIC SYNTHESIS OF SELENONEINE AND ITS ANALOGS

(71) Applicant: The Trustees of Princeton University, Princeton, NJ (US)

(72) Inventors: Mohammad R. SEYEDSAYAMDOST, Princeton, NJ (US); Chase M. KAYROUZ, Plainsboro, NJ (US)

(73) Assignee: The Trustees of Princeton University, Princeton, NJ (US)

(21) Appl. No.: 18/849,848

(22) PCT Filed: Mar. 24, 2023
(86) PCT No.: PCT/US2023/016183

§ 371 Date: Sep. 23, 2024

Related U.S. Application Data
(60) Provisional application No. 63/325,533, filed on Mar. 30, 2022.

(51) Int. Cl. C12P 17/10; C12N 9/02; C12N 9/10; C12N 9/12

(52) U.S. Cl. CPC C12P 17/10; C12N 9/0083; C12N 9/1048; C12N 9/1294; C12Y 114/99; C12Y 207/09003

(57) ABSTRACT

Disclosed is a method for forming selenoneine or analogs thereof. The method may include phosphorylating sodium selenide to a selenophosphate, using adenosine triphosphate (ATP) and at least a first protein, generating a selenosugar by converting the selenophosphate using at least a second protein in the presence of a common sugar donor, and forming selenoneine or an analog thereof by combining the selenosugar with N,N,N-trimethyl-L-histidine or analogs thereof using at least a third protein. The method may include combining SenA, SenB, and SenC in an aqueous buffer at neutral pH and ambient temperature, and allowing SenA, SenB, and SenC to form selenoneine or an analog thereof in the presence of ATP, a common sugar donor, and sodium selenide.

### CROSS-REFERENCE TO RELATED APPLICATIONS

The present application claims priority to U.S. Provisional Patent No. 63/323,533, filed Mar. 25, 2022, the contents of which are incorporated by reference herein in its entirety.

### STATEMENT REGARDING FEDERALLY SPONSORED RESEARCH OR DEVELOPMENT

This invention was made with government support under Grant No. GM140034 awarded by the National Institutes of Health. The government has certain rights in the invention.

### SEQUENCE LISTING

The application contains a Sequence Listing which has been submitted electronically in ST.26 Sequence listing XML format and is hereby incorporated by reference in its entirety. Said ST.26 Sequence listing XML, created on Mar. 9, 2023, is named PRIN-87976.xml and is 13,877 bytes in size.

### TECHNICAL FIELD

The present application is drawn to techniques for synthesizing selenoneine and its analogs, and chemoenzymatic synthesizing of those compounds in particular.

### BACKGROUND

Selenoneine and its analogs are important antioxidants with vitamin-like and therapeutic properties. When compared to its well-commercialized sulfur analog, ergothioneine, selenoneine exhibits an enhanced radical scavenging activity, methylmercury detoxification functionality, and resistance to oxidative degradation.

The myriad cytoprotective properties of selenoneine have been known for some time. However, how cells synthesize this molecule has remained elusive. Methods have been developed for the chemical production of selenoneine. However, these utilize harsh chemicals as the reactive element selenium must be incorporated in a specific fashion.

### BRIEF SUMMARY

Various deficiencies in the prior art are addressed below by the disclosed techniques and kits.

In various aspects, a method for forming selenoneine or analogs thereof may be provided. The method may include phosphorylating sodium selenide to a selenophosphate, using adenosine triphosphate (ATP) and at least a first protein. The method may include generating a selenosugar (which may be, e.g., 1-seleno-N-acetyl-β-D-glucosamine) by converting the selenophosphate using at least a second protein in the presence of a common sugar donor (which may be, e.g., UDP-glucose). The method may include forming selenoneine or an analog thereof by combining the selenosugar with N,N,N-trimethyl-L-histidine or an analog thereof using at least a third protein. The method may include purifying the selenoneine or analog thereof. The first protein, second protein, and/or third protein are fused to an affinity tag. The first protein may be coded by a sell homolog, and may encode, e.g., SenC. The second protein may be a glycosyltransferase, such as, e.g., SenB. The third protein may be coded by an egtB homolog, and may encode, e.g., SenA. The third step-forming selenoneine or an analog thereof by combining the selenosugar with N,N,N-trimethyl-L-histidine—may include forming hercyncyl-SeGlcNAc selenoxide (GlcNAc-SEN=O), and allowing hercyncyl-SeGlcNAc selenoxide (GlcNAc-SEN=O) to spontaneously convert to selenoneine or be reduced to hercynyl-SeGlcNAc seleno-ether (GlcNAc-SEN).

In various aspects, an alternate method for forming selenoneine or an analog thereof may be provided. The method may include combining SenA, SenB, and SenC in an aqueous buffer at neutral pH and ambient temperature. The method may then include allowing SenA, SenB, and SenC to form selenoneine or an analog thereof in the presence of ATP, a common sugar donor, and sodium selenide.

In various aspects, a kit may be provided. The kit may include one or more vectors comprising (i) a first gene coding for at least SenC; (ii) a second gene coding for at least SenB; and (iii) a third gene coding for at least SenA. The kit may optionally include adenosine triphosphate, a common sugar donor, sodium selenide, and/or hercynine or an analog thereof.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIGS. 1 and 2 are flowcharts of a method for generating selenoneine or an analog thereof.

FIG. 3A is an illustration showing representative sen gene clusters in selected bacteria.

FIG. 3B is an illustration of the three-gene sen cluster consisting of the egtB homolog senA, a member of a superfamily senB, and a selD homolog senC.

FIG. 4 is an illustration showing the ergothioneine biosynthetic pathway.

FIG. 5 is an illustration showing Reactions shown to be carried out by SenC and SenB; SenB is shown as accepting three different UDP-sugars indicated to generate selenoglucose (SeGlc), seleno-N-acetylglucosamine (SeGlcNAc), or seleno-Nacetylgalactosamine (SeGalNAc).

FIG. 6 is an illustration and graph showing a diselenide product of the SenB reaction.

FIG. 7 is an illustration showing a mBBr derivatization reaction.

FIG. 8 is an illustration showing the structure of the mBBr-derivatized SeGlc along with relevant 1H-13C HMBC NMR correlations (arrows) used to solve the structure.

FIG. 9 contains logo plots displaying multiple sequence alignments of SenA and EgtB proteins; amino acids are numbered with respect to the structurally characterized EgtB from M. thermoresistibile. The catalytic tyrosine and the iron-binding residues are conserved. Some divergence is observed in the thiol- and hercynine-binding residues.

FIG. 10 is an illustration showing the biosynthetic pathway for selenoneine (SEN) with the reactions disclosed herein.

FIG. 11 is an illustration showing an internal Cope elimination to form selenoneine.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Disclosed is chemoenzymatic synthesis of selenoneine and its analogs. Disclosed and identified herein are the genes required for selenoneine production in diverse microbes. Using these, a facile, green, cell-free chemoenzymatic synthesis of selenoneine can be shown.

The disclosed process can be used to generate large amounts of selenoneine and its analogs. The process is ‘green’ in that it uses enzymes for key transformations rather than harsh chemicals. With this approach, variants of selenoneine may be generated and tested in diverse assays.

Using a computational approach, microbial genome sequences were searched for possible genes that may be involved in selenoneine biosynthesis. Clusters of 3-5 genes (depending on the organism) were identified, which have been named the sen cluster. It was shown that the sen cluster generates selenoneine using two heretofore unknown carbon-selenium bond-forming reactions. The minimal cluster encodes the enzymes SenA, SenB, and SenC. SenC is a SelD analog; it uses ATP to convert sodium selenide to selenophosphate. Next, SenB—a protein belonging to a novel and previously uncharacterized family of enzymes-uses selenophosphate to generate 1-seleno-β-D-glucose, the first biosynthetic pathway for the production of a selenosugar. Other products include 1-seleno-N-acetyl-β-D-glucosamine, or 1-seleno-N-acetyl-β-D-galactosamine. Finally, in another heretofore unknown and unusual reaction, SenA combines the selenosugar (i.e. 1-seleno-N-acetyl-β-D-glucosamine) with N,N,N-trimethyl-L-histidine to produce selenoneine. SenC, SenB, and SenA were used in a one-pot reaction to complete selenoneine synthesis in vitro in aqueous buffer at neutral pH and ambient temperature.

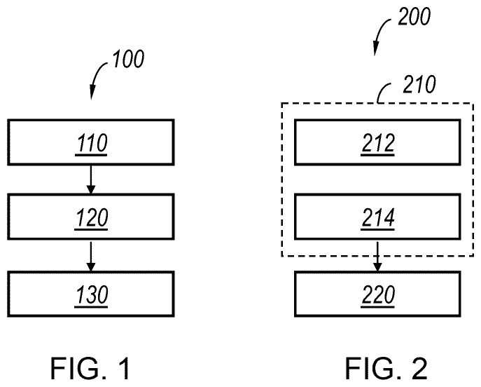

In various aspects, a method for producing selenoneine or an analog thereof may be provided. Referring to FIG. 1, the method 100 may include phosphorylating 110 sodium selenide to a selenophosphate, using adenosine triphosphate (ATP) and at least a first protein.

The first protein may be encoded by a selD homolog. As used herein, the term “homolog” refers to a gene (or a nucleic acid sequences derived therefrom or comprised by said gene) related to a second gene (or such nucleic acid sequence) by descent from a common ancestral DNA sequence. The term, “homolog” includes genes separated by the event of speciation (“ortholog”) and genes separated by the event of genetic duplication (“paralog”). With respect to sequence homology, in some embodiments, sequences may be homologs if they are at least 60%, preferably at least 70%, more preferably at least 80%, more preferably at least 90%, more preferably at least 95% identical, more preferably at least 97% identical, or more preferably at least 99% identical.

The selD homolog may code for SenC [SEQ ID NO. 1] or a homolog thereof. The first protein may be fused to an affinity tag. Any appropriate affinity tag may be used here. As used herein, the term “affinity tag” refers to a peptide enabling a specific interaction with a specific ligand. In some embodiments, the affinity tag is linked to a molecule said combination being referred to herein as a “fusion molecule” or “fusion construct”. Accordingly, the present invention relates to a fusion molecule comprising the affinity tag as described herein. The affinity tag can be linked directly or indirectly to said molecule. The tag can be linked to any site of the molecule, e.g. to or near the end or terminus of the molecule, to one or more internal sites, attached to a side chain, or to the amino-terminal amino acid (N-terminal) or to the carboxy-terminal amino acid (C-terminal). Also more than one affinity tag can be linked to the molecule.

In some embodiments, a DNA sequence encoding the first protein and an affinity tag may be, e.g., 6×His-senC [SEQ ID NO. 2] or a homolog thereof.

The method may include generating 120 a selenosugar by converting the selenophosphate (from the phosphorylating step) using at least a second protein in the presence of a common sugar donor.

The second protein may be a glycosyltransferase. As used herein, the term “glycosyltransferase” refers to an enzyme capable of catalyzing the transfer of sugar moieties from activated donor molecules to specific acceptor molecules, forming glycosidic bonds. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org). In some embodiments, the glycosyltransferase may be SenB [SEQ ID NO. 3] or a homolog thereof. In some embodiments, the second protein may be fused to an affinity tag as disclosed herein. In some embodiments, a DNA sequence encoding the second protein and an affinity tag may be, e.g., 6×His-senB [SEQ ID NO. 4] or a homolog thereof.

The common sugar donor may be any non-GDP-based common sugar donor, and is preferably a UDP-based common sugar donor. Non-limiting examples of such donors include, e.g., uridine diphospho-D-glucose (UDP-glucose or UDP-Glc), uridine diphospho-D-galactose (UDP-galactose or UDP-Gal), uridine diphospho-D-xylose (UDP-Xyl), uridine diphospho-N-acetyl-D-glucosamine (UDP-GlcNAc), uridine diphospho-N-acetyl-D-galactosamine (UDP-GalNAc), uridine diphospho-D-glucuronic acid (UDP-GlcA), and uridine diphospho-D-galactofuranose (UDP-Galf).

Non-UDP common sugars that may function include cytidine monophospho-N-acetylneuraminic acid (CMP-Neu5Ac), and cytidine monophospho-2-keto-3-deoxy-D-mannooctanoic acid (CMP-Kdo).

The selenosugar may be, e.g., 1-seleno-N-acetyl-β-D-glucosamine.

The method may include forming 130 selenoneine or an analog thereof by combining the resulting selenosugar from the previous step with N,N,N-trimethyl-L-histidine or an analog thereof using at least a third protein. As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar to or comparable in function and appearance to the reference compound.

The third protein may be encoded by an egtB homolog. In some embodiments, the egtB homolog may code for SenA [SEQ ID NO. 5] or a homolog thereof. In some embodiments, the third protein may be fused to an affinity tag as disclosed herein. In some embodiments, a DNA sequence encoding the second protein and an affinity tag may be, e.g., 6×His-senA [SEQ ID NO. 6] or a homolog thereof.

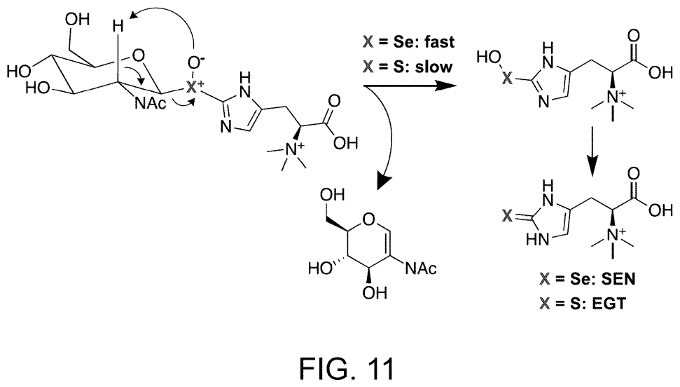

In some embodiments, this third step may include forming hercyncyl-SeGlcNAc selenoxide (GlcNAc-SEN=O), and allowing hercyncyl-SeGlcNAc selenoxide (GlcNAc-SEN=O) to spontaneously convert to selenoneine (see FIG. 11) or be reduced to hercynyl-SeGlcNAc seleno-ether (GlcNAc-SEN).

In various aspects, a particular method for forming selenoneine or an analog thereof may be provided. Referring to FIG. 2, the method 200 may include combining 210 various components (e.g., in a container, such as a reaction vessel) This may include combining 212 SenA [SEQ ID NO. 5], SenB [SEQ ID NO. 3], and SenC [SEQ ID NO. 1] in an aqueous buffer at neutral pH and ambient temperature. In some embodiments, the neutral pH may be a pH of, e.g., 6-8, and may preferably be a pH of 7-8, and more preferably from 7-7.4. In some embodiments, the ambient temperature may be 20-30° C.

The method may include introducing 214 adenosine triphosphate (ATP), a common sugar donor, and sodium selenide to the buffer. This step may also include introducing hercynine or an analog thereof to the buffer. As will be understood, the combination steps can be performed in any order. The method may include allowing 220 SenA, SenB, and SenC to form selenoneine or an analog thereof in the presence of adenosine triphosphate (ATP), a common sugar donor, and sodium selenide.

In various aspects, a kit may be provided. The kit may include one or more vectors comprising (i) a first gene coding for at least SenC; (ii) a second gene coding for at least SenB; and (iii) a third gene coding for at least SenA. The kit may optionally include adenosine triphosphate, a common sugar donor, sodium selenide, and/or hercynine or an analog thereof.

### Example

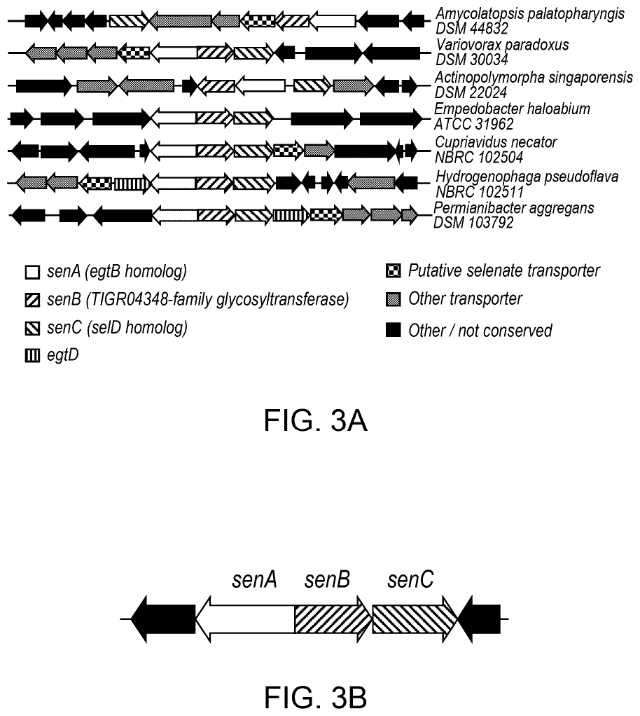

A metabolomic analysis was performed of two species that harbor the selD-egtB-tigr04348 cluster. The actinomycete Amycolatopsis palatopharyngis DSM 444832 and the β-proteobacterium Variovorax paradoxus DSM 30034 were grown in the presence of sodium selenite (see FIG. 3A).

The cells were then pelleted and analyzed by HPLC-MS, revealing the production of ergothioneine and its selenium-analog, selenoneine, by both bacteria.

Specifically, for various tested species, ISP Medium 2 agar plates and tryptic soy broth were used for general maintenance and liquid cultures of Amycolatopsis palatopharyngis DSM 44832 and Streptomyces rimosus ATCC 10970. Nutrient agar plates and nutrient broth were used for general maintenance and liquid cultures of Variovorax paradoxus DSM 30034. LB agar and broth (supplemented with 50 mM MOPS, pH 7.0) were used for general maintenance and liquid cultures of Burkholderia thailandensis E264.

For each strain, a single colony from an agar plate was inoculated into a sterile culture tube containing 5 mL of liquid medium and incubated at 30° C./250 rpm. The starter cultures were then used to inoculate 5 mL liquid cultures supplemented with 100 μM of filter-sterilized Na₂SeO₃ and incubated at 30° C./250 rpm. Production cultures of B. thailandensis were grown for 24 hours, V. paradoxus and S. rimosus for 48 hours, and A. palatopharyngis for 72 hours. Following incubation, 2 mL of each culture was transferred to an Eppendorf tube, pelleted by centrifugation, and supernatants were removed. Cell pellets were resuspended in 0.4 mL of ddH₂O and subjected to hot-water extraction by heating to 95° C. for 15 minutes. After cooling to room temperature, the mixtures were pelleted by centrifugation and supernatants were analyzed by HPLC-MS. Analytes were separated on a Synergi™ Fusion-RP HPLC column (from Phenomenex, 100×4.6 mm, 4 μm) with a flow rate of 0.5 mL/min and an elution program consisting of 0% solvent B wash for 5 min, a linear gradient from 0-100% B over 10 min, followed by a hold at 100% B for 3 min.

Upon further genomic examination, both strains were found to harbor a canonical ergothioneine BGC (egt) in addition to the putative selenometabolite BGC. To examine whether selenoneine may be the product of nonspecific Se incorporation by the egt cluster, two relatives of the producing strains, the actinomycete Streptomyces rimosus ATCC 10970 and the β-proteobacterium Burkholderia thailandensis E264, which encode a canonical egt cluster but lack the putative selenometabolite cassette, were analyzed in the same fashion, revealing exclusive production of ergothioneine, and not selenoneine. These observations are consistent with prior reports of the inability of the bacterial ergothioneine biosynthetic machinery to generate selenoneine in vitro. These results suggest that selenoneine may in fact be the product of the new cluster, which is herein termed sen, with senA, senB, and senC coding for an egtB homolog, the putative glycosyltransferase, and a selD homolog, respectively (see FIG. 3B).

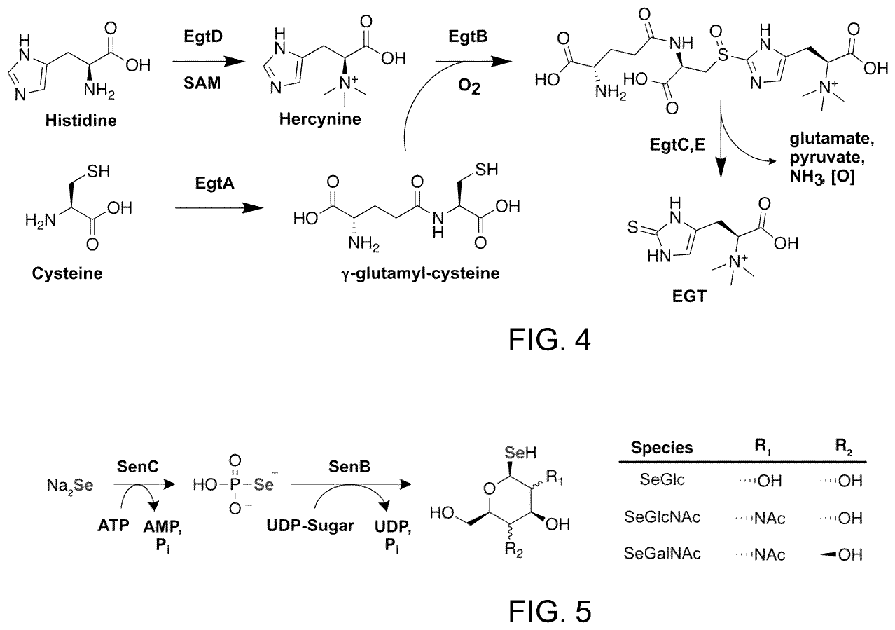

This indicates two distinct biosynthetic routes to ergothioneine and selenoneine. Ergothioneine is produced by egtABCDE. Early steps involve EgtA-catalyzed isopeptide bond formation between cysteine and glutamic acid, and S-adenosylmethionine (SAM)-dependent trimethylation of histidine by EgtD [SEQ ID NO. 7]. See FIG. 4. The products, γ-glutamyl-cysteine (GGC) and hercynine, are then oxidatively linked via a sulfoxide by the atypical nonheme iron enzyme EgtB. Finally, following EgtC-catalyzed hydrolysis of the glutamic acid residue, EgtE performs PLP-dependent C—S bond cleavage to furnish ergothioneine.

In contrast, the sen cluster contains only three genes. It is believed that SenB utilizes SeP (generated by SenC) to generate a new selenometabolite. SenA then catalyzes C—Se bond-formation between this new species and hercynine, followed by C—Se bond-cleavage to give selenoneine. Hercynine would likely be siphoned from the canonical ergothioneine pathway, though a small subset of sen clusters feature a co-localized egtD gene.

To evaluate this proposal and conclusively link the production of selenoneine to the sen cluster, the entire biosynthetic pathway was reconstructed in vitro. SenA, SenB, SenC, and EgtD were cloned from V. paradoxus and isolated as soluble, His-tagged proteins through heterologous expression in E. coli. For example, 6×His-EgtD [SEQ ID NO. 8] was generated to encode for EgtD [SEQ ID NO. 7] fused to a his-tag. See Tables 1 and 2, below.

TABLE 1
Primers used in this example.

| Primer | Sequence (5′->3′) | Purpose |
|---|---|---|
| Vpa-SenC-F | tggtgccgcgcggcagccatatg | Amplification |
| | aacgctgccctccc | of senC for |
| | [SEQ ID NO. 9] | assembly into |
| Vpa-SenC-R | agtggtggtggtggtggtgctca | pET-28b(+) |
| | gcggatcaccagcc | |
| | [SEQ ID NO. 10] | |
| Vpa-EgtD-F | tggtgccgcgcggcagccatatg | Amplification |
| | cgcaatccaagctcc | of egtD for |
| | [SEQ ID NO. 11] | assembly into |
| Vpa-EgtD-R | agtggtggtggtggtggtgctca | pET-28b(+) |
| | agcccccaaccaatg | |
| | [SEQ ID NO. 12] | |

TABLE 2
Plasmids used or generated in this example.

| Plasmid | Purpose | Source |
|---|---|---|
| pET-28b(+) | KanR, Expression vector | Novagen |
| pET-28b(+) 6xHis-SenA | Expression of 6xHis-SenA | This example |
| pET-28b(+) 6xHis-SenB | Expression of 6xHis-SenB | This example |
| pET-28b(+) 6xHis-SenC | Expression of 6xHis-SenC | This example |
| pET-28b(+) 6xHis-SenD | Expression of 6xHis-EgtD | This example |

For the plasmids, genomic DNA from Variovorax paradoxus DSM 30034 was isolated using the WIZARD® Genomic DNA Purification Kit (from Promega) following the manufacturer's instructions. From genomic DNA, senC and egtD genes were PCR-amplified using Q5 High Fidelity DNA polymerase (NEB) with primers Vpa-SenC-F/R and Vpa-EgtD-F/R, respectively, which have overhangs that allowed for assembly into pET28b (+) (Table S3). SenA and senB from Variovorax paradoxus DSM 30034 were obtained as synthetic DNA fragments, codon-optimized for expression in E. coli and with overhangs to allow assembly into pET28b (+). Protein expression plasmids were assembled from gene fragments and vector pET28b (+), linearized with NdeI and XhoI (from NEB), using NEBUILDER® HiFi DNA Assembly Master Mix (from NEB) following the manufacturer's instructions. Ligation mixtures were transformed into chemically-competent E. coli DH5α by heat-shock and plated onto LB agar containing 50 mg/L kanamycin. After confirmation by Sanger sequencing, assembled plasmids were transformed into E. coli BL21 (DE3) for protein expression.

For expression and purification of 6×His-tagged SenA, SenB, SenC, and EgtD proteins, all four proteins were produced separately in E. coli BL21 (DE3) cells grown in two 4 L flasks, each containing 2 L Terrific Broth supplemented with 50 mg/L kanamycin at 37° C./170 rpm. Small cultures were prepared by inoculating 40 mL of LB medium containing 50 mg/L Kan with a single colony of E. coli BL21 (DE3) carrying the desired plasmid. After overnight growth at 37° C./170 rpm, 4 L of TB medium plus 50 mg/L Kan were inoculated with the 40 mL small culture and incubated at 37° C./170 rpm. At OD600=0.5-0.6, protein expression was induced with 0.2 mM IPTG, and cultures were incubated at 37° C./170 rpm for an additional 12-24 hours. Cells were pelleted by centrifugation (8,000 g, 15 min, 4° C.), yielding ˜7 g of cell paste per L. The cell pastes were stored at −80° C. until purification.

All purification steps were carried out in a cold room at 4° C. Cells were resuspended in lysis buffer (5 mL/g cell paste), which consisted of 25 mM Tris-HCl, 300 mM NaCl, 10 mM imidazole, 10% glycerol, pH=7.7, supplemented with 1 μL/mL Protease Inhibitor Cocktail (from Sigma) and 1 mM phenylmethylsulfonyl fluoride. Once homogenous, 0.1 mg/mL deoxyribonuclease I (from Alfa Aesar) was added, and the cells were lysed by the addition of 5 mg/mL lysozyme followed by sonication using 30% power (˜150 W) in 15 s on/15 s off cycles for a total of 4 min. This process was repeated twice. The lysate was then clarified by centrifugation (17,000 g, 15 min, 4° C.) and loaded onto a 5 mL Ni-NTA column pre-equilibrated in lysis buffer. The column was washed with lysis buffer and His-tagged proteins were eluted with elution buffer consisting of 25 mM Tris-HCl, 300 mM NaCl, 300 mM imidazole, 10% glycerol, pH=7.7. Eluted proteins were then buffer-exchanged using a 50 mL column of SEPHADEX® G-25 medium (from Cytiva) into storage buffer consisting of 25 mM Tris-HCl, 150 mM NaCl, 10% glycerol, pH=7.7. Purified proteins were stored at −80° C. Protein concentrations were determined spectrophotometrically on a CARY® 60 UV-visible spectrophotometer (from Agilent) using calculated molar extinction coefficients at 280 nm. From 4 L cultures, the following yields were obtained: 105 mg SenA, 198 mg SenB, 152 mg SenC, and 63 mg EgtD.

As expected, SenC was found to catalyze the ATP-dependent phosphorylation of sodium selenide to yield SeP. See FIG. 5.

The selenophosphate synthetase activity of SenC was characterized according to known, conventional methods. Under anaerobic conditions, a 700 μL reaction containing 2 mM dithiothreitol (DTT), 1 mM ATP, 1.5 mM Na₂Se, and 20 μM SenC was prepared in buffer consisting of 50 mM tricine, 20 mM KCl, 5 mM MgCl₂, 10% D₂O, pH=7.2. Control reactions were prepared in an identical fashion, lacking either SenC or Na₂Se, or with Na₂S in place of Na₂Se. After a 1-hour incubation period at room temperature, the reactions were transferred to NMR tubes, removed from the glovebox, and immediately analyzed by ³¹P-NMR. Results were consistent with previous reports of selenophosphate synthetase enzymes, with conversion to selenophosphate (SeP) only observed in the presence of all components. No production of thiophosphate was observed in the presence of Na₂S.

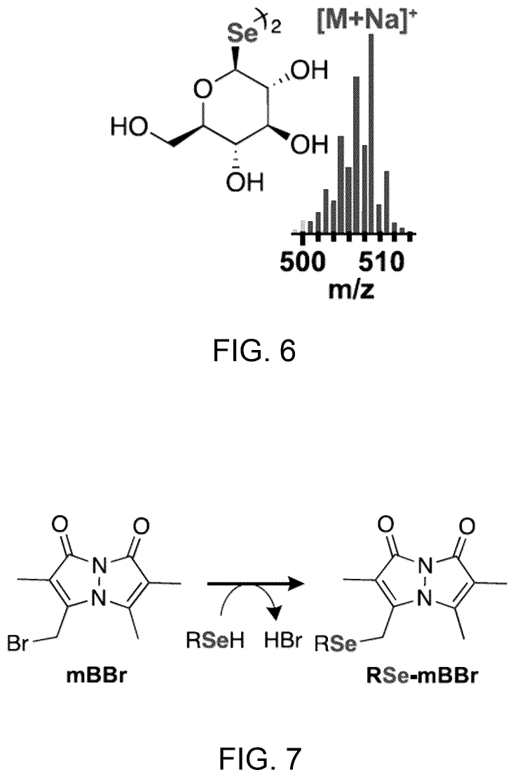

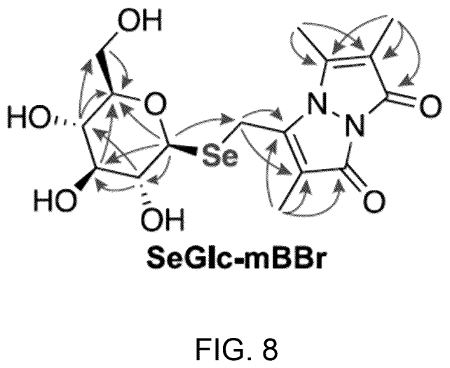

Next, the function of SenB, the C-terminus of which shows weak homology to NDP-sugar binding domains of glycosyltransferases, was evaluated. When SenB was added to the SenC reaction along with the common sugar donor UDP-glucose, production of a new species was observed. It contained two Se atoms, as judged by the characteristic Se isotope pattern obtained by HR-MS, implying a potential diselenide-dimer. See FIG. 6. Derivatization of the species with the thiol-labeling reagent monobromobimane (mBBr) resulted in an mBBr-derivative with a single Se atom and a mass consistent with the monomeric form of the underivatized species. See FIG. 7. Purification of the mBBr-derivative and structural elucidation by multidimensional NMR spectroscopy revealed 1-seleno-β-D-glucose (SeGlc), a rare example of a naturally-occurring selenosugar, as the SenB reaction product. See FIG. 8.

Specifically, in vitro reconstitution of SenB activity was performed as follows. Under anaerobic conditions, 200 μL reactions containing 2 mM DTT, 2 mM ATP, 1 mM Na₂Se, 20 μM SenC, 20 μM SenB, and 2 mM of NDP-sugar were prepared in buffer consisting of 50 mM tricine, 20 mM KCl, 5 mM MgCl₂, pH=7.2. Control reactions were prepared in an identical fashion, lacking either SenB, SenC, or ATP. After a 6-hour incubation period at room temperature, reactions were removed from the glovebox and exposed to oxygen for 30 minutes to oxidize any unreacted Na₂Se. Next, 50 μL of each reaction mixture was quenched with 50 μL of MeOH, while another 50 μL was quenched with 50 μL of 10 mM mBBr in MeCN. Reactions were incubated for an additional 30 min at room temperature in the dark to allow for complete derivatization with mBBr. Reactions quenched with MeOH were filtered and analyzed by LC-MS using a KINETEX® Polar C18 column (from Phenomenex, 150×4.6 mm, 2.6 μm) with a flow rate of 0.4 mL/min and an elution program consisting of 0-20% solvent B over 5 min, followed by 20-100% solvent B over 3 min, and a final step of 3 min at 100%. This assay demonstrated the production of underivatized selenosugar diselenides. Reactions quenched with mBBr were filtered and analyzed by LC-MS using a SYNERGI® Hydro-RP HPLC column (from Phenomenex, 250×4.6 mm, 4 μm) with a flow rate of 1 mL/min and an elution program consisting of a 5% solvent B wash for 3 min, a gradient of 5-75% solvent B over 6 min, followed by a gradient of 75-100% solvent B over 1 min, and a final hold at 100% for 5 min. The remaining 100 μL of each reaction mixture was lyophilized for use as a crude selenosugar substrate in downstream assays with SenA.

SenB enzymatic assays were carried out anaerobically as described above on a 10-mL scale with UDP-glucose (Glc) and UDP-N-acetylglucosamine (GlcNAc). Reactions were quenched with 3 mL of 10 mM mBBr in MeCN and incubated on a platform rocker for 1 hour in the dark to facilitate complete derivatization. SeGlc-mBBr was purified by semi-preparative HPLC using a SYNERGI® Fusion-RP HPLC column (from Phenomenex, 250×10 mm, 4 μm) with a flow rate of 2.5 mL/min and elution consisting of a gradient of 0-40% solvent B for 15 min, followed by 40-100% solvent B for 3 min, and a final hold at 100% B for 5 min. SeGlcNAc-mBBr was purified by semi-preparative HPLC using a LUNAR C18 column (from Phenomenex, 250 ×10 mm, 5 μm) with a flow rate of 2.5 mL/min and elution consisting of a gradient of 10-50% solvent B for 10 min, followed by a gradient of 50-100% solvent B for 10 min, followed by a hold at 100% for 3 min. Purified compounds were dissolved in DMSO-d6 and analyzed by NMR spectroscopy, confirming the presence of a selenium atom bound to the anomeric carbon, which was determined to be ß-configured for both selenosugars, as shown by the large axial-axial coupling constants between protons on the sugar C-1 and C-2 (9.2 Hz for SeGlc-mBBr and 10.5 Hz for SeGlcNAc-mBBr).

Control assays further confirmed the requirement of each reaction component, and in the absence of SenB, no SeGlc was formed. Controls lacking either SenC or ATP also abolished SeGlc production, verifying SeP as the source of Se for SenB. Moreover, no product was observed with sodium sulfide, suggesting SenC discriminates between Se and S, thus synthesizing only SeP for the downstream SenB-catalyzed reaction. When assayed against various nucleotide-sugar substrates, SenB was found to efficiently utilize UDP-N-acetylglucosamine (UDP-GlcNAc) and UDP-N-acetylgalactosamine (UDP-GalNAc), but not GDP-mannose or GDP-glucose, suggesting SenB may be specific for UDP-sugars.

Together, these results allow the designation of SenB as a novel selenosugar synthase, which now joins SelA and SelU as only the third bona fide Se—C bond-forming enzyme characterized to date.

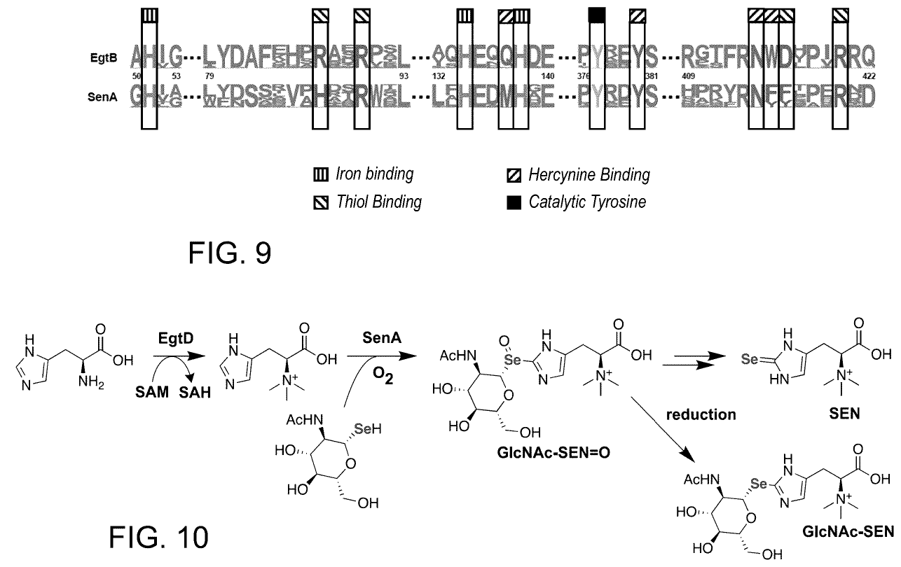

The remaining enzyme encoded in the sen cluster, SenA, is a distant homolog of the C—S bond-forming sulfoxide synthase EgtB. Previous work by others has provided extensive characterization of this family of nonheme iron enzymes, including crystal structures that have pinpointed residues involved in iron-, hercynine-, and thiol-binding. SenA proteins bear 30-50% similarity to members of the EgtB family and share its conserved iron-binding three-His facial triad, catalytic Tyr residue, and motifs involved in hercynine-binding. See FIG. 9.

However, a conserved Arg87/Asp416 pair implicated in thiol binding within the EgtB active site is replaced with His and Phe or Tyr, respectively, in all SenA proteins. These substitutions suggest that SenA catalyzes Se—C bond-formation between hercynine and a different substrate, presumably a selenosugar, en route to selenoneine.

This hypothesis was tested by first recapitulating the activity of V. paradoxus EgtD in vitro, allowing for rapid formation of hercynine from histidine and SAM. See FIG. 10, which shows the biosynthetic pathway for selenoneine (SEN) with the reactions described in this example. Hercyncyl-SeGlcNAc selenoxide (GlcNAc-SEN=O) can spontaneously convert to selenoneine or be reduced to hercynyl-SeGlcNAc selenoether (GlcNAc-SEN).

For in vitro reconstitution of EgtD activity, under aerobic conditions, 100 μL reactions containing 0.5 mM histidine, 2 mM S-adenosylmethionine (SAM), and 20 μM EgtD were prepared in buffer consisting of 50 mM Tris-HCl, 150 mM NaCl, pH=8.0. A control reaction lacking EgtD was prepared in an identical fashion. After a 30-minute incubation period at room temperature, reactions were quenched with 100 μL MeOH, filtered, and analyzed by LC-MS using a SYNERGI® Hydro-RP HPLC column (from Phenomenex, 250×4.6 mm, 4 μm) with a flow rate of 1 mL/min and an elution program gradient consisting of a 0% solvent B wash for 3 min, a followed by a gradient to 100% solvent B over 2 min, and a hold at 100% for 5 min. Results were consistent with previous reports of EgtD enzymes, with conversion of histidine to hercynine observed only in the presence of EgtD.

For in vitro reconstitution of SenA activity, under aerobic conditions, crude selenosugar substrates from lyophilized SenBC assay mixtures were first dissolved in buffer consisting of 50 mM Tris-HCl, 150 mM NaCl, pH=8. Next, 0.5 mM histidine, 2 mM S-adenosylmethionine (SAM), 2 mM DTT, 0.2 mM (NH₄)₂Fe(SO₄)₂, 20 μM EgtD, and 20 μM SenA were added to a final volume of 100 μL. Control assays were prepared in an identical fashion, except lacking either EgtD or SenA. Assays containing sodium β-D-thioglucose (from Fisher) in place of selenosugar substrate were also prepared in a similar fashion. However, thioglucose was found to be a very poor substrate for SenA, and thus 80 μM enzyme was used to facilitate appreciable conversion. After a 7-hour incubation period at room temperature, 50 μL of each reaction mixture was quenched with 50 μL of MeOH, while another 50 μL was quenched with 50 μL of 10 mM mBBr in MeCN. Reactions were incubated for an additional 30 minutes at room temperature in the dark to allow for complete derivatization with mBBr. The samples were then filtered and analyzed by LC-MS using a SYNERGI® Hydro-RP HPLC column (from Phenomenex, 250×4.6 mm, 4 μm) with a flow rate of 1 mL/min and elution consisting of 5% solvent B for 3 min, a gradient of 5-75% solvent B over 6 min, followed by a gradient of 75-100% over 1 min, and a hold at 100% for 5 min.

For structural characterization of SenA products, a large-scale SenA enzymatic assay was carried out aerobically as described above using a crude SeGlcNAc substrate from a lyophilized, 10-mL SenBC reaction mixture. Reactions were quenched with 5 mL of 10 mM mBBr in MeCN and incubated on a platform rocker for 1 hour in the dark to facilitate complete derivatization. Selenoneine-mBBr (SEN-mBBr) was purified by semi-preparative HPLC using a LUNA® C18 HPLC column (from Phenomenex, 250×10 mm, 5 μm) with a flow rate of 2.5 mL/min and an elution program consisting of a gradient of 7.5-80% solvent B for 12 min, followed by a hold at 80% for 4 min. Purified SEN-mBBr was dissolved in D₂O and analyzed by NMR spectroscopy. The selenium atom was confirmed to be positioned at the imidazole C2 carbon, as evidenced by the absence of an imidazole C-2 proton and diagnostic H-13C HMBC correlations. The structures of other SenA products (hercynyl-SeGlcNAc selenoether [GlcNAc-SEN], hercynyl-SeGlc selenoether [Glc-SEN], hercynyl-thioglucose sulfoxide [Glc-EGT=O], and selenoneine [SEN]) were confirmed by tandem HR-MS/MS analysis.

Upon incubation of SenA with hercynine (generated in situ with EgtD, His, and SAM), various selenosugars, dithiothreitol (DTT), and Fe(II), followed by derivatization with mBBr, a new species was observed containing a single Se atom and a high-resolution mass consistent with that of selenoneine-mBBr. The structure was confirmed by NMR spectroscopy upon purification of the product from large-scale reactions with SeGlcNAc, the substrate for which SenA showed the greatest preference. More importantly, in the absence of mBBr, selenoneine itself was directly observed in the product mixture as evidenced by HR-MS and HR-MS/MS analysis, thus completing reconstitution of the entire selenoneine biosynthetic pathway in vitro and confirming that the sen cluster is responsible for building this unusual natural product.

In ergothioneine biosynthesis, the EgtB reaction yields hercynyl-GGC sulfoxide, which is further processed by EgtC and EgtE to produce ergothioneine. See FIG. 4. In this example, the analogous product by SenA, hercynyl-SeGlcNAc selenoxide, was not detected in the above-described reactions. However, further examination of the mBBr-derivatized reaction revealed the reduced form of this product, hercynyl-SeGlcNAc selenoether; its structure was corroborated by HR-MS and HR-MS/MS analysis. It is believed this product forms as a result of reduction of the selenoxide intermediate by excess DTT in the reaction, implying that the selenoxide intermediate is indeed formed. Spontaneous selenoxide eliminations are well-documented, and this intermediate could undergo, aside from reduction by DTT, rapid internal Cope elimination to form the selenenic acid (see FIG. 11), which would convert to selenoneine through either reduction or disproportionation. Selenoxide internal eliminations occur upwards of 10⁵ times more rapidly than the related sulfoxide eliminations. Thus, the tendency of selenoxides to undergo internal elimination obviates the need for an EgtE-like reaction in selenoneine biosynthesis. In support of this proposal, replacement of SeGlcNAc with commercially available thioglucose in the SenA reaction mixture resulted in accumulation of hercynyl-thioglucose sulfoxide, with no observed formation of ergothioneine or reduction to the corresponding thioether.

As seen, the process disclosed herein utilizes neutral-pH aqueous buffer and ambient temperature for the production of selenoneine. The disclosed approach could therefore be used for rapid, ‘green’ production of selenoneine and its analogs.

What is claimed:

1. A method for forming selenoneine or analogs thereof, the method comprising:
phosphorylating sodium selenide to a selenophosphate, using adenosine triphosphate (ATP) and at least a first protein;
generating a selenosugar by converting the selenophosphate using at least a second protein in the presence of a common sugar donor; and
forming selenoneine or analog thereof by combining the selenosugar with N,N,N-trimethyl-L-histidine or analog thereof using at least a third protein.

2. The method according to claim 1, wherein the common sugar donor is UDP-glucose.

3. The method according to claim 1, wherein the selenosugar is 1-seleno-N-acetyl-β-D-glucosamine.

4. The method according to claim 1, wherein the first protein is coded by a selD homolog, the second protein is a glycosyltransferase, and the third protein is coded by an egtB homolog.

5. The method according to claim 1, wherein the first protein is SenC, the second protein is SenB, and the third protein is SenA.

6. The method according to claim 1, wherein forming selenoneine or an analog thereof by combining the selenosugar with N,N,N-trimethyl-L-histidine includes forming hercyncyl-SeGlcNAc selenoxide (GlcNAc-SEN=O), and allowing hercyncyl-SeGlcNAc selenoxide (GlcNAc-SEN=O) to spontaneously convert to selenoneine or be reduced to hercynyl-SeGlcNAc seleno-ether (GlcNAc-SEN).

7. The method according to claim 1, further comprising purifying the selenoneine or analog thereof.

8. The method according to claim 1, wherein the first protein, second protein, and/or third protein are fused to an affinity tag.

9. A method for forming selenoneine or an analog thereof, the method comprising:
combining SenA, SenB, and SenC in an aqueous buffer at neutral pH and ambient temperature; and
allowing SenA, SenB, and SenC to form selenoneine or an analog thereof in the presence of ATP, a common sugar donor, and sodium selenide.

10. A kit comprising:
one or more vectors, comprising:
a first gene coding for at least SenC;
a second gene coding for at least SenB;
a third gene coding for at least SenA;
optionally adenosine triphosphate;
optionally a common sugar donor;
optionally sodium selenide; and/or
optionally hercynine or an analog thereof.


SEQUENCE LISTING (8 sequences)

SEQ ID NO: 1 - protein, 367 aa, Variovorax paradoxus
SSGLVPRGSHMNAALPQAPAAEPRLTSLSHGGGCGCKIAPGVLSEILKGTARMPMPPELLVGIETADDAAVYQLNDEQALIATTDFFMPIVDDPFDFGRIAATNAISDVYAMGGKPILALALVGMPINVLSTATIGRILEGGESVCRAAGIPIAGGHTIDSVEAIYGLVALGLVHPKRVKRNAGAKAGDLLVLGKPLGVGILSAALKKEALDAAGYQRMIATTTRLNTSGPELAALDGVHALTDVTGFGLAGHALELARGAKLSLEIDWQQVPVLEGVRGLVQSGHVTGASGRNWAGYGSDVALPQNFAEEERALLTDPQTSGGLLVSCAPEALDEVLAIFRRHGFDDAAVIGTASAQGSAPRLVIR

SEQ ID NO: 2 - DNA, 1134 bp, synthetic construct
  source 1..1134: mol_type = other DNA
atgggcagcagccatcatcatcatcatcacagcagcggcctggtgccgcgcggcagccatatgaacgctgccctcccgcaagcccccgccgccgagccccgcctcacctcgctgtcgcacggcggcggctgcggctgcaagatcgcgcccggcgtgctgtcggaaatcctcaagggcacggcgcgcatgccgatgccgcccgagctgctggtcggcatcgagacggccgacgacgcggccgtctaccagctcaacgacgagcaggcgctgatcgccaccaccgacttcttcatgccgatcgtcgacgatccgttcgacttcggccgcatcgcggccaccaacgcgatctccgacgtgtatgcgatgggcggcaagccgatcctggcgctcgcgctggtcggcatgccgatcaacgtgctcagcacggcgaccatcgggcgcatcctcgaaggcggcgaatcggtgtgccgggcggcgggcattccgatcgcgggcggccacaccatcgattcggtcgaggccatctacgggctggtcgcgctcggcctggtccacccgaagcgcgtcaagcgcaatgccggcgccaaggccggcgacctgctggtgctgggcaagccgctgggcgtgggcatcctgtcggcggcgctcaagaaggaggcactcgacgcggccggctaccaacgcatgatcgccaccacgacgcggctgaacacgtccggccccgagctcgcggcgctcgacggcgtgcatgcgctgaccgacgtgacgggcttcggcttggccggccatgcgctggaactggcacgcggcgcaaagctgtcgctggagatcgactggcagcaggtgcccgtgctggaaggcgtgcgcggcctggtgcagtcgggacacgtcaccggcgcatccggccgcaactgggccggctacggctccgatgtggcgctgccgcagaactttgccgaggaagagcgcgccctgctcaccgacccgcagaccagcggcggactgctggtttcgtgcgcgcccgaggcgctcgacgaggtgctggccatcttccgccggcatggcttcgacgacgccgcggtgattggcactgccagcgcgcaagggagcgcgccgcggctggtgatccgctga

SEQ ID NO: 3 - protein, 341 aa, Variovorax paradoxus
SSGLVPRGSHMSNPSLVIVSPALPGANNGNWRTAQRWKALLSPVCSARVVQQWPDADASADTVMLALHARRSAESIAHWAHAHPGRGLGVVLTGTDLYQDIGSDPQAQRSLQLAQRLVVLQALGAEALPPECRAKARVVYQSTSARAELPKSARQLRAVMVGHLRQVKSPQTLFDAARLLCGREDIRIDHIGDAGDAGLGELARALASDCPGYRWLGALPHAQTRQRIQRAHVLVHTSALEGGAHVIMEAVRSGTPVLASRVPGNVGMLGNDYAGYFPHGDAAALAALLEACRAGQGSKDRAAGLLDSLRTQCALRAPLFDPRAEQAALFQLLNELQPPPP

SEQ ID NO: 4 - DNA, 1056 bp, synthetic construct
  source 1..1056: mol_type = other DNA
atgggcagcagccatcatcatcatcatcacagcagcggcctggtgccgcgcggcagccatatgagcaacccgagcctggtgattgtgagcccggcgctgccgggcgcgaacaacggcaactggcgcaccgctcaacgctggaaagcgctgctgagccctgtgtgctccgcacgcgttgtgcagcagtggccggatgcggatgcgagcgcggataccgtgatgctggcgctgcatgcgcgccgcagcgcggaaagcattgcgcattgggcgcatgcgcatccgggccgcggcctgggcgtggtgctgaccggcaccgatctgtatcaagatatcggtagcgaccctcaagcgcagaggtcactgcagctggcgcagcgactggtggtgttgcaagcgctgggcgcggaagctttacccccggaatgccgcgcgaaagcccgcgtggtgtatcagagcacgagcgcgcgtgcggaactgccgaaaagcgcgcgtcagctgcgcgcggtgatggtgggccatctgcgccaagtgaaaagcccgcagaccctgtttgatgcggcgcgcctgctgtgcggccgcgaagatattcgcattgatcatattggggacgcgggcgatgccggccttggagaactcgcgcgtgcgcttgcgtcagattgccctgggtacagatggttgggcgcgctgccgcatgcgcagacccgtcagcgcattcagcgcgcgcatgtgctggtgcatacgagcgcgctggaaggcggcgcgcatgtgattatggaagcggtgcgcagcggcaccccggtgctggcgagccgcgtgccgggcaacgtgggcatgctgggcaacgattatgcgggctattttccgcatggtgatgcggcggcactggcggcactgctggaggcgtgccgcgcgggccaagggagcaaagatcgcgcagcgggcttactggattcgctgcgcacgcagtgcgcgctccgtgcgccgctgtttgatccgcgcgcggaacaagcggcgctgtttcagctgctgaacgaactgcagccgccgccgccgtaa

SEQ ID NO: 5 - protein, 426 aa, Variovorax paradoxus
SSGLVPRGSHMDSTLPVYSVAGAPEALALRAGPPASVRAALLAARRRTLDLADDFRAALGDAYPGIGYAPELNPPLWELGHVAWFQEWWIGRNRQRARGVACEPDHAREPSLLPQADAWYDSGRVAHRTRWALPLPDAEATRDYLERTLAQTLALLDELPPDAHDDALYFFRLVALHEAMHAEAAAYMAEGLGIALREGGVAPQLAEDAELELPAQRLRMGSDAGTGFAFDNELLSHDVSIEPLRIDAQAVSWARFLPFVEAGGYEHPAWWSDAGRDWLARQLLRHPAHLRAAGTGWQQRRGGRWLPLDPQGAAVHLNAHEAEAWCRWAGRRLPTEAEWECAALTLPGFAWGRVWEWTSSPFEPYPGFAPHPYRDYSAPWFGTRRVLRGACHATSAALAHARYRNFFEPHRRDIFAGFRSCRAPGG

SEQ ID NO: 6 - DNA, 1312 bp, synthetic construct
  source 1..1312: mol_type = other DNA
atgggcagcagccatcatcatcatcatcacagcagcggcctggtgccgcgcggcagccatatggacagtaccttaccggtgtattcagttgctggcgcgcccgaagcgttagcattgcgcgcgggaccccctgcgagtgtgcgggctgcgcttctcgccgcacgccgccgcaccctggatctggcggatgattttcgtgcggctctgggagacgcgtatccgggcattggctatgcgccggaactgaacccgccgctgtgggaactgggccatgtggcgtggtttcaagaatggtggattggccgaaaccgtcagcgcgcacgcggagtggcgtgcgaaccggatcatgcgcgcgaaccgagcctgctgccgcaagcggatgcttggtatgactccggccgcgtagcgcatcgcacgcgctgggctctgccgctgccggatgcggaagcgacccgcgattatctggaacgcaccctggcgcagaccctggcgctgctggatgaactgccgccggatgcgcatgatgatgcgctgtatttttttcgcctggtggcgctgcatgaagcgatgcatgcggaagcggcggcgtatatggcggaaggcctgggcattgcgctgcgcgaaggcggcgtggcgccgcagctggcggaagatgcggaactggaactgccggcgcagcgcctgcgcatgggcagcgatgctgggaccggatttgcgtttgataacgaactgctgagccatgatgtgagcattgaaccgctgcgcattgatgcgcaagcggtgagctgggcgcgctttctgccgtttgtggaagcgggcggctatgaacatccggcatggtggtcagatgcgggacgcgattggctggcgcggcaactgctgcgccatcctgcgcatsttacgggcggctggcaccggctggcagcagagacgtgggggccgttggctgccactggatccgcaaggcgcggcggtgcatttaaatgctcacgaagccgaagcgtggtgtcgctgggcgggacgccgactcccgaccgaagcggaatgggaatgcgcggcgctgaccctgccgggcttcgcgtggggacgcgtgtgggaatggacgagcagcccgtttgaaccgtatcccggcttcgcgccacacccgtatcgcgattatagcgcgccgtggtttggcacccgcagagtgctgcgcggcgcgtgtcatgcgacgagcgcggcgctggcccacgcgcgctatcgcaacttttttgaaccgcatcgccgcgatatttttgcgggctttcgcagctgccgcgcgccgggcggctaa

SEQ ID NO: 7 - protein, 349 aa, Variovorax paradoxus
SSGLVPRGSHMRNPSSSSSFSFAAAQPQQRQPVAPAAAEKNAALLSEFGREMQAGLARRPRSISPKYFYDAAGSQLFDRICELPEYYPTRTELRILGECAGEIAAQVGPGAEIVEFGAGSLTKVRLLLDALEAPRRYLPIDISGEHLAAAAERLRADYPQLAVQPIAADYTMPLVLPAPLAGAGKRVGFFPGSTIGNFEPDEALAFLQLAARMLRGGGLLIGVDLVKDPGRLHAAYNDAQGVTAAFNLNLLRRANAELDANFDLEGFAHAAFYNAPKQRIEMHLVSRRDQIVSLNGERFAFEEGETIHTEYSHKFTVEGLRALAVKAGFRPGAVWTDPERLFSVHWLGA

SEQ ID NO: 8 - DNA, 1080 bp, synthetic construct
  source 1..1080: mol_type = other DNA
atgggcagcagccatcatcatcatcatcacagcagcggcctggtgccgcgcggcagccatatgcgcaatccaagctcctcgtcctccttttcgtttgccgccgcccagccgcagcagcgccagcctgtcgcgcctgccgccgcggaaaaaaatgcggcgctgctctccgagttcggccgcgagatgcaggcgggcctcgcgcgcaggccgcgcagcatttcgcccaagtatttttacgacgccgcgggttcgcagctgttcgaccgcatctgcgagctgcccgagtactaccccacgcgcaccgagctgcgcattcttggcgaatgcgcgggcgagatcgccgcgcaggtgggtccgggcgccgagatcgtcgagttcggcgccggctcgctcaccaaggtgcggctgctgctcgatgcgctcgaggcgcccaggcgctacctgccgatcgacatttccggcgagcacctggcggccgccgccgagcggctcagggccgactacccgcagctggcggtgcagccgatcgcggcggactacaccatgccgctggtgctgcccgcgccgctcgccggtgcgggcaagcgcgtgggttttttccccggctcgacgatcggaaatttcgagcccgacgaggcactggccttcctgcagctcgcggcgcgcatgctgcgcggcggcgggctgctgatcggcgtcgacctggtgaaggacccgggccgcctgcatgcggcctacaacgacgcgcagggcgtgaccgcggcgttcaacctcaacctgctgcggcgcgccaacgccgagctcgacgcgaacttcgatctcgagggctttgcgcacgcggcgttctacaacgcgccgaaacagcgcatcgagatgcacctggtgagccggcgggaccagatcgtttcgctgaacggcgagcggtttgcgttcgaggaaggggagaccattcacaccgagtactcgcacaagttcactgtggaaggattgcgggcgctggctgtgaaggcggggttccggccgggggctgtgtggacggatccggagaggctcttcagcgtgcattggttgggggcttga